(19) United States
(12) Patent Application Publication
(10) Pub. No.: US 2002/0166149 A1
(43) Pub. Date: Nov. 7, 2002

(54) Sustained totipotent regenerable tissue culture of Arundo donax (Giant Reed) and totipotent tissue and plants produced therefrom

(76) Inventors: Laszlo Marton, Chapin, SC (US); Mihaly Czako, Columbia, SC (US)

Correspondence Address: Nelson Mullins Riley & Scarborough LLP, Keenan Building, Third Floor, 1330 Lady Street, Columbia, SC, 29201, US

(21) Appl. No.: 10/068,600

(22) Filed: Feb. 5, 2002

Related U.S. Application Data
(60) Provisional application No. 60/266,068, filed on Feb. 5, 2001.

(51) Int. Cl. A01H 1/00; C12N 15/82; C12N 15/87; A01H 5/00; C12N 5/00; C12N 5/02

(52) U.S. Cl. 800/320; 800/278; 435/430.1

(57) ABSTRACT

A method is provided for generating sustained totipotent tissue cultures of giant reed (Arundo donax L.), and for micropropagating giant reed in vitro, wherein immature inflorescence are cultivated to produce totipotent tissue which is suitable for sustained maintenance and propagation. Greening of the tissue can be induced under light and the multishoot culture can multiply by microtillering. Foreign genes can be introduced into the tissue if desired, and the transgenic plants can be used in phytoremediation technologies in the field and in phytoreactors independently of seasons.

### CROSS REFERENCE TO RELATED PATENTS AND PATENT APPLICATIONS

The present application claims the benefit of copending provisional application 60/266,068, filed Feb. 5, 2001, which is relied on herein and hereby incorporated herein by reference in its entirety.

### BACKGROUND OF THE INVENTION

(1) Field of the Invention

The present invention relates to a method for the production of plants of giant reed (Arundo donax) on a large scale, and more particularly to a method for the production of cloned plants of Arundo donax with the potential for the production of transgenic Arundo donax plants, and to the plants produced by the method.

(2) Description of the Related Art

Arundo donax L., or Giant Reed, of the Family Poaceae (Gramineae), is one of the largest grasses in the world, and is an attractive, robust, perennial reed. Tucker, G. C., J. Arnold Arb., 71:145-177 (1990). The very strong, somewhat woody, clustering culms, which grow from horizontal knotty rootstocks, are known to grow to a height of 8-10 meters and to have a diameter of from 1 to 4 cm. Bailey, L. H., Manual of cultivated plants: Most commonly grown in the continental United States and Canada, Rev. Ed., MacMillan, New York, (1954); and Mabberley, D. J., The plant-book: a portable dictionary of the vascular plants, 2^{nd} Rev., Cambridge Univ. Press, Oxford (1997). It is one of the largest of the herbaveous grasses and has fleshy, creeping rootstocks that form compact masses from which arise tough fibrous roots that penetrate deeply into the soil. The culms commonly branch during the second year of growth and are hollow with walls of 2 to 7 mm thick.

The plant is known by a variety of common names, including carrizo, bamboo reed, Danubian reed, donax cane, Italian reed, Provence cane and Spanish reed. A. donax probably originated from the freshwaters of the warm regions of eastern Asia. It has been in cultivation in Asia, North Africa, and the Middle East for thousands of years and also in North and South America, Australia and South Africa, during the past century. Further information on the culture of A. donax can be found, for example, in Bell, G. P. Ecology and management of Arundo donax, and approaches to riparian habitat restoration in Southern California, in Plant Invasions: Studies From North America and Europe, Brock, J. H. et al., Eds. pp.103-113, Backhuys Publishers, Leiden (1997); Perdue, R. E., Econ. Bot., 12:368-404 (1958); Rossa, B. et al., Bot. Acta, 111:216-221 (1998); Roys, R., Ethnobotany of the Maya: The Department of Middle American Research, M.A.R. Series Pub. 2, Tulane U., New Orleans (1931); Zahran, M. A. et al., The vegetation of Egypt, Chapman & Hall, London (1992); and Zohary, M., Plant Life of Palestine, Ronald Press, New York (1962).

A. donax is a multipurpose plant. It has been used for 5,000 years for pipe instruments and is the source for reeds for clarinets and organ pipes. Even with today's modern technology, most of the reeds for woodwind musical instruments are still made from a. donax culms.

Giant reed is also used for erosion control and has great potential for use as an energy crop. Szabo, P., et al., J. Anal. Appl. Pyrolysis, 36:179-190 (1996). The culms are also used for fishing rods, walking sticks, mats and lattices in the construction of adobe hits. Giant reed is also a source of industrial cellulose for paper and rayon making, and for the production of other polysaccharides. Neto, C. P. et al., Ind. Crops & Prods., 6:51-58 (1997). It has even been considered as a source of pulp for the making of paper. Perdue, R., Arundo donax: Source of Musical Reeds and Industrial Cellulose, www.wuarchive.wustl.edu/doc/misc/org/doublereeds/general/cane.html.

Giant reed grows very rapidly. When conditions are favorable, growth at a rate of 0.3 to 0.7 meter per week for several weeks is not unusual. Young culms typically grow to their full diameter within the initial growing season, but their walls increase in thickness thereafter. Id.

Outside its native range and the Mediterranean, however, the plant is sterile; it flowers, but does not produce viable seed. It reproduces vegetatively efficiently from fragments of stems and rhizomes. Boose, A. B. etal., Weed Res., 39:117-127 (1999). Traditional horticultural propagation of giant reed is by division of rhizomes. However, the propagation of giant reed by either rhizome division, or by traditional seed culture require a significant amount of time and effort between the initiation of division, or planting, and the successful establishment of a growing plant. Moreover, conventional methods of propagation provide limited opportunity for genetic manipulation, and, in the case of seeds, do not permit genetic control of the resulting progeny. Such conventional techniques also require large areas for the production of a sufficient number of plants to be useful in programs for the production of fuel or biomass, or for use in bioremediation programs.

Accordingly, it would be useful to be able to provide a method by which A. donax could be propagated even in areas in which it is sterile and in a manner that would require shorter time, less effort and less area than conventional methods. In particular, it would be useful if a method could be provided that permitted better genetic manipulation and control of the plants. Moreover, it would also be useful if the method was independent of seasons and was sustainable at a high rate of propagation.

### SUMMARY OF THE INVENTION

Briefly, therefore the present invention is directed to a novel method for the production of totipotent tissue culture of giant reed (Arundo donax L.), the method comprising: selecting an explant of living tissue from Arundo donax L.; and cultivating the A. donax tissue on a primary medium to produce totipotent A. donax tissue culture.

The present invention is also directed to a novel method for the micropropagation of giant reed (Arundo donax L.), the method comprising: selecting an explant of living tissue from Arundo donax L.; cultivating the A. donax tissue on a primary medium to produce a totipotent tissue culture; cultivating the totipotent A. donax tissue on a secondary medium to produce complete plantlets having roots and shoots; and acclimating the plantlets in soil.

The present invention is also directed to novel totipotent Arundo donax tissue that is produced by the method described first above.

The present invention is also directed to novel transgenic totipotent Arundo donax tissue that is produced by the method described first above, but with the additional step of adding a heterologous gene to the A. donax tissue.

The present invention is also directed to a novel plant of Arundo donax L. that is produced by the method described second above.

The present invention is also directed to a novel transgenic plant of Arundo donax L. that is produced by the method described second above, but with the additional step of adding a heterologous gene to the A. donax tissue.

The present invention is also directed to a novel method for removal of an environmental pollutant from wastewater, the method comprising: providing at least 10 A. donax plants that possess the same genetic characteristics; establishing the plants in a liquid medium; and contacting the roots of the plants in the liquid medium with an environmental pollutant, thereby causing the environmental pollutant to be removed from the liquid medium.

The present invention is also directed to a novel method for bioremediation of an environmental pollutant from a land area, the method comprising: providing at least 10 A. donax plants that possess the same genetic characteristics; establishing the plants in soil; and contacting the roots of the plants with the environmental pollutant in the land area, thereby causing the environmental pollutant to be removed from the land area.

Among the several advantages found to be achieved by the present invention, therefore, may be noted the provision of a method by which A. donax can be propagated even in areas in which it is sterile. Such method also provides for propagation that can be carried out in a manner that would require shorter time, less effort and less area than conventional methods. Such method also provides for better genetic manipulation and control of the plants. The novel method also provides for the ability to carry out these activities in a manner that is independent of seasons and is sustainable at a high rate of propagation.

### BRIEF DESCRIPTION OF THE DRAWINGS

FIG. 1 is a photograph of in vitro regenerating cell cultures of Arundo donax L. produced from unemerged A. donax influrescence explants at four different stages of development, where plate A contains green callus forming at the tips of pedicels and inflorescence stem segments and from flower parts after four weeks under light on DM-8 medium; plate B shows etiolated shoots forming from the primary callus in the dark after six weeks on DM-8 medium; plate C shows sustained culture on DM-8 medium under light; and plate D shows sustained culture on DM-8 medium in the dark; and

FIG. 2 is a photograph of plants six weeks after they were transferred to potting soil and which are clones of A. donax that were grown by the present method from totipotent A. donax culture tissue.

### DETAILED DESCRIPTION OF THE PREFERRED EMBODIMENTS

In accordance with the present invention, it has been discovered that regenerable tissue can be produced from A. donax tissues by a method wherein the tips of field-grown or greenhouse grown pre-flowering shoots with leaf sheaths completely enclosing the developing but yet unemerged immature inflorescence, whose surface has been sterilized, are stripped of the leaves and the inflorescences are cut into cross-sectional pieces, which are then cultivated on a solid-type primary medium containing plant hormones. Multishoot formation, but not elongation, occurs on the primary medium, and so the method is therefore suitable for sustained maintenance and propagation of the totipotent tissue culture.

As used herein, the term “totipotent” means having unlimited capability to produce any type of cell. Totipotent cells have the capability to turn (or “specialize”) into all of the tissues and organs that are present in the completely developed plant. In other words, totipotent cells have the capability to regenerate into whole plants.

Another aspect of the present invention is a method for regeneration of complete plantlets with roots and partially elongated shoots which continue to multiply by microtillering on a solid-type secondary medium containing a plant hormone.

A further aspect of the invention is a method for inducing shoot elongation on a solid-type tertiary medium containing no plant hormones.

The totipotent tissue culture is suitable for introduction of foreign genes by means of cocultivation of the cross-sectional pieces of inflorescences with Agrobacterium tumefaciens, or by the biolistic and other direct DNA transfer methods of injecting heterologeous genetic material into the totipotent regenerable tissue culture. Suitable techniques for the genetic engineering of A. donax are described, for example, in Barcelo, P. et al., Advances in Botanical Research Incorporating Advances in Plant Pathology, 34:59-126 (2001); Christou, P., Particle bombardment methods in cell biology, 50:375-382 (1995); Christou, P., Field Crops Res., 45(1-3):143-151 (1996); and Christou, P., Trends in Plant Sci., 1(12):423-431 (1996).

In general, the present method includes the following steps: An explant of living tissue of A. donax is obtained. The explant is cultivated in a primary cultivation step in which totipotent tissue is generated. In a preferred embodiment, greening is induced in the totipotent tissue generated in the primary cultivation step by subjecting the tissue to light. The totipotent tissue that is generated in the primary cultivation step is then cultivated in a secondary cultivation step in which complete plantlets are induced. The plantlets can then be transferred to soil for acclimation. It is preferred, however, that, after the secondary cultivation, the plantlets are cultivated in a tertiary cultivation step to permit shoot elongation prior to transfer to soil. When the plantlets have become acclimated in soil, they can be transplanted to any desired location, including the location for final planting.

When an explant of living tissue from A. donax is obtained for use in the present method, the tissue can be living A. donax tissue that is obtained from any source. The genetic material can be obtained from a living A. donax plant, or it can be obtained as tissue culture, or any other tissue, from any one of the steps of the present method.

When the explant is obtained from a living A. donax plant, it is preferred that it is obtained from an immature inflorescence. An example of a starting material for the explant of the present invention can be obtained from the tips of field-grown or greenhouse-grown pre-flowering shoots with leaf sheaths completely enclosing the developing, but yet unemerged immature inflorescence. It has been found that an immature inflorescence enclosed in leaf sheaths before blooming is preferred since it exhibits a higher yield of regenerable tissue than other tissue sources.

To prepare the explant for cultivation, all but the terminal leaf sheaths are carefully stripped so as not to expose the inflorescence. The shoot tips can then be sanitized, or surface sterilized. One method of surface sterilization is by immersing the shoot tips in a solution of 5×diluted commercial bleach containing 10% v/v ethanol and 0.1% Tween 80 surfactant for 15 minutes. The shoot tips can then be rinsed three times with sterile water prior to further use. Such sterilization reduces or eliminates environmental bacterial contamination.

The inflorescence is then excised from all leaf sheaths under aseptic conditions and is cut into cross-sectional pieces. Any sterilized sharp blade, knife, or scalpel can be used for this step. By cutting an aseptic immature inflorescence containing a number of meristematic regions into cross-sectional pieces, the formation of regenerable tissue is induced.

The pieces of the cut-up inflorescence is then cultivated in a primary cultivation step in which totipotent tissue is generated. It is preferred that the primary cultivation be carried out in the dark and at approximately room temperature. It is also preferred that the cultivation be carried out on a solid-type medium that contains plant hormones. The duration of the primary cultivation step is sufficiently long for multishoot tissue formation, but not elongation, to occur. It is preferred that the primary cultivation step have a duration of from about two weeks to about six weeks, and even more preferred that it has a duration of about four weeks, yet more preferred, that the primary cultivation step have a duration of four weeks.

For A. donax tissue, a preferred temperature range for the primary cultivation step is from about 15° C. to about 35° C., a temperature range of about 20° C. to about 30° C. is more preferred, a temperature of about 26° C. to 28° C. is even more preferred, and a temperature of about 25° C. is yet more preferred.

The medium that is useful for the primary cultivation step can be a basal medium for plant tissue culture. Examples of suitable medium include, without limitation, DM-8 medium (as described below), or MS medium, or Gamborg's B5 medium at full or ½ strength. It is preferred that the primary medium is supplemented with a plant hormone. Examples of suitable plant hormones include auxins, such as 2,4-dichlorophenoxyacetic acid, and picloram. In preferred embodiments, these hormones can be employed in combination with cytokinins, such as benzyladenine, zeatin, or thidiazuron.

One example of a medium for the primary cultivation step can be prepared by adding to sterile water MS (Murashige and Skoog, 1975) basal salts (Sigma Fine Chemicals), 4.3 g/l; Miller's salt solution (6% w/v KH₂PO₄), 3 ml;-myo-inositol, 100 mg/l; Vitamix (Marton and Browse, 1991), 2 ml; sucrose, 30 g/l; all mixed into water, supplemented with the plant growth regulators adenine hemisulfate, 80 mg/l; picloram, 0.12 mg/l; indole-3-butyric acid, 1 mg/l; 2,4-dichlorophenoxyacetic acid, 0.5 mg/l; isopentenyladenine, 0.5 mg/l; trans-zeatin, 0.5 mg/l; and thidiazuron 3 mg/l; and solidified with Phytagel (Sigma Fine Chemicals), 2 g/l.

It is preferred that a gellant, such as Gellan gum, for example, Phytagel, available from Sigma Co., St.Louis, Mo., is also employed in the medium at conventional rates. Less purified Gellan substitutes, such as Gelcarin, agarose, or agar can also be used.

It is preferred that the pH of the medium for the primary cultivation step is adjusted to 5.8 before the medium is sterilized. By way of example, the medium can be sterilized in a pressure cooker for 25 minutes at a temperature of about 109° C. and at a pressure of about 35 kPa.

The warm medium may be poured into a sterile petri dish and allowed to cool to room temperature. The cut-up genetic material can then be distributed upon the surface of the gelled medium, and the petri dish covered with a lid to preserve sterility. The covered dish can then be placed in a location suitable for maintaining the temperature as discussed above.

It is also preferred that the tissue being cultured is kept in the dark during the primary cultivation step. However, as an alternative, the genetic material may be subjected to continuous illumination during the primary cultivation step. If continuous illumination is employed, it is preferred that it be of an intensity of about 30-50 μmol m⁻²s⁻¹, and be a mixture of incandescent and cool white fluorescent tubes.

During the primary cultivation step, multishoot formation occurs from the cut-up explant tissue, but without shoot elongation. The culture at this point comprises totipotent tissue (which may also referred to herein as totipotent, or regenerable, tissue culture). The totipotent tissue can then be moved forward to the secondary cultivation step, or it can be used as the genetic material for initiation of another cycle of primary cultivation. Therefore, the totipotent tissue culture can be used as a regenerable source of genetic material for sustained maintenance and propagation.

In a preferred embodiment, greening of the etiolated dark-grown tissue produced in the primary cultivation step may be initiated under light in about three days in the culturing room with artificial illumination.

After completion of the primary cultivation step, the totipotent tissue is then cultivated in a secondary cultivation step in which complete plantlets are induced. The medium that is useful for the secondary cultivation step can be a basal medium for plant tissue culture such as MS, or Gamborg's B5 medium at full or ½ strength. it is preferred that the medium is supplemented with a plant hormone. Examples of plant hormone that can be employed include cytokinins, such as benzyladenine, zeatin, and thidiazuron.

In one example, the medium for the secondary cultivation is prepared by adding to sterile water from about 0.01 to about 1 mg/l, preferably about 0.02 ml/l, of a cytokinin, such as thidiazurone, 30 g/l of sucrose, and about 3 ml of Miller's salt solution (6% w/v KH₂PO₄). The medium can be gelled and sterilized as described for the primary medium.

Totipotent tissue from the primary cultivation step is then used to inoculate the secondary medium. The inoculated secondary cultivation medium is then cultured, either in the dark or under continuous light, at about room temperature, for a period of from about one week to about four weeks. During the cultivation, it is preferred that the growing tissue multiplies by microtillering. As used herein, tillers are lateral branches that form at below ground nodes. Tillering is a term used for branching of wheat and other cereal crops, or grasses in general. When used in to describe tissue growth in micropropagation (in vitro propagation) the term “microtillering”, means a plant response in vitro involving perpetual branching or formation of a sustained multishoot culture. At the end of the secondary cultivation, the culture will contain complete plantlets with roots and partially elongated shoots.

At this point, the plantlets can be either moved directly to soil for acclimation, or they can be cultivated in a tertiary cultivation step to permit shoot elongation prior to transfer to soil.

It is preferred that the plantlets are moved into a tertiary medium that is similar to the medium that is used for the secondary cultivation step, but containing no plant hormones. The tertiary cultivation step is carried out at substantially room temperature, and for a duration of about four weeks.

The plantlets are then transferred from the tertiary medium to soil for acclimation.

When the plantlets have become acclimated in soil, they can be transplanted to any desired location, including the location for final planting.

Some of the properties that make giant reed so attractive for phytoremediation are the phenomenal growth rate of up to 6.3 cm per day, and fast regeneration after cropping. As mentioned above, A. donax attains heights more than 4 meters in less than one growing season. This growth rate is supported by an unusually high photosynthetic capacity (maximum photosynthetic CO₂ uptake between 19.8 and 36.7 μmol m⁻²s⁻¹), and a very large water use (2,000 l/m² of standing A. donax). A. donax can produce up to 100 tons per hectare of above-ground biomass. In North America and other locations, it forms pure stands because of the lack of natural predators and competitors. It does not provide habitat or food for wildlife because it contains chemicals that protect if from insects and grazers. It can grow in water, and can oxidize sulfides and reduce heavy metal ions by releasing oxygen into the anaerobic organic sediment. The fibrous roots of the creeping rootstock penetrate up to 4.9 m deep in sand. A. donax thrives in both alkaline and acidic conditions in mild drainage and absorbs heavy metals in a pH dependent manner.

A. donax has been utilized in constructing wetlands for agricultural waste treatment (in combination with other species), and for the treatment of municipal wastewater. The ability to culture and regenerate A. donax will allow genetic transformation to be applied to the species. It then may be possible to generate transgenic variants for example with increased phytoremediation potential.

Among the advantages of the present method is the ability to obtain high frequency plant regeneration from immature inflorescences. Sustainable multiple shoot cultures have been established from giant reed where shoot elongation and rooting are controlled by the type and concentration of plant growth regulators in the synthetic culture medium. The in vitro grown plantlets are established easily in the soil.

Furthermore, it is believed that the efficiently produced plant clones can also be utilized for scientific research in physiology and genetics. A. donax tissues, at different stages of the in vitro propagation, are suitable for introduction of foreign genes. After such genetic modification, it should be possible to regenerate complete transgenic plants, and then to clonally propagate such transgenic individuals by this method. These efficient, large-scale micropropagation techniques would permit genetically modified clones of A. donax to be available in large numbers for industrial applications such as phytoremediation technologies in the field or in bioreactors.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

### EXAMPLE 1

This illustrates the formation of complete A. donax plantlets from excised tissue and shows the effect of different media upon shoot and root development.

Shoot tips bulging with developing inflorescences were collected from a large, naturalized patch of giant reed in the Southeastern United States in August. All but one of the leaf sheaths were stripped carefully so as not to expose the inflorescence. Shoot tips were disinfected by shaking in a solution of mercuric chloride plus 0.1% Tween 80 surfactant for 15 minutes. Shoot tips were rinsed three times with sterile water. The immature inflorescences were excised, chopped and placed on DM-8 or II₁-S medium in the dark or under continuous illumination (30-50 μmol m⁻²s⁻¹, composed of a mixture of incandescent and fluorescent tubes—Sylvania and Power Twist Vita-Lite 40 W) at 26° C. to 28° C.

Callus was cultured every four weeks for maintenance. Regenerated and rooted plants were separated, potted in the greenhouse, and initially kept under plastic wrap cover to help acclimation.

DM-8 medium contained MS (Murashige and Skoog, 1975) basal salts (Sigma Fine Chemicals), 4.3 g/l; Miller's salt solution (6% w/v KH₂PO₄), 3 ml; myo-inositol, 100 mg/l; Vitamix (Marton and Browse, 1991), 2 ml; sucrose, 30 g/l; all mixed into water, supplemented with the plant growth regulators adenine hemisulfate, 80 mg/l; picloram, 0.12 mg/l; indole-3-butyric acid, 1 mg/l; 2,4-dichlorophenoxyacetic acid, 0.5 mg/l; isopentenyladenine, 0.5 mg/l; trans-zeatin, 0.5 mg/l; and thidiazuron 3 mg/l; and solidified with Phytagel (Sigma Fine Chemicals), 2 g/l.

DM-3 medium differed only in the plant growth regulators, which were: adenine hemisulfate, 10 mg/l; 2,4-dichlorophenoxyacetic acid, 0.2 mg/l; thidiazuron, 0.1 μM.

DM-5 contained MS salts, 4.3 g/l; sucrose 30 g/l; thidiazuron, 0.1 μM.

Hormone-free medium was the same as DM-5, but without thidiazuron.

II₁-S medium contained MS basal salts, 4.3 g/l; (NH₄)₂SO₄, 200 mg/l; Miller's salt solution, 3 ml; myo-inositol, 200 mg/l; Vitamix, 2 ml; L-glutamine, 200 mg/l; sucrose, 30 g/l; mixed into sterile water, supplemented with the plant growth regulator, 2,4-dichlorophenoxyacetic acid, 1 mg/l; and solidified with agar (granulated, Fisher Scientific, Fair Lawn, N.J.), 2 g/l.

The pH of all tissue culture media was adjusted to 5.8 before sterilization in a pressure cooker at 109° C., 35 kPa pressure, for 25 minutes.

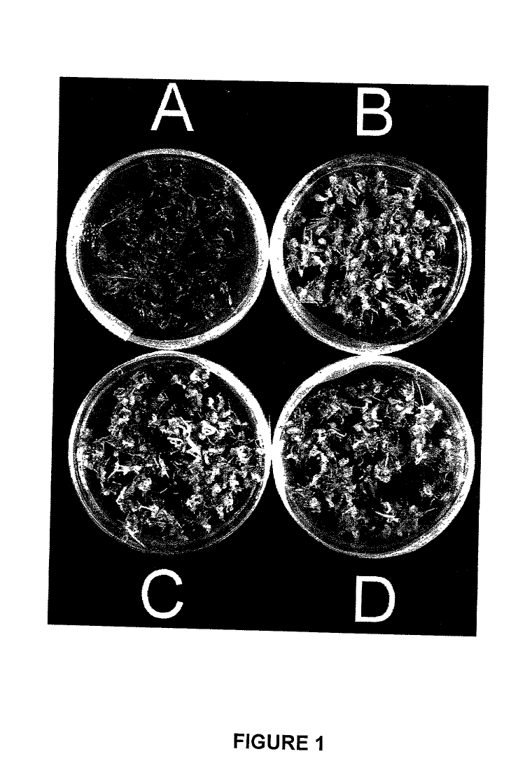

Sterile shoot tips containing immature inflorescences were cut into small cross-sectional segments (1-3 mm) and placed on two different media—ll₁-S and DM-8. In three to four weeks, callus formed at the tips of pedicels and inflorescence stem segments and from flower parts (See FIG. 1A). Callus on II₁-S medium was white and more or less translucent, without any sign of differentiation, and was not studied further. Callus on DM-8 medium was white and more or less translucent, initially without shoots, but soon displaying signs of differentiation and pale yellowish color. On DM-8 medium, etiolated shoots formed from the primary callus in the dark if left on the original medium for 4 to 6 weeks. (FIG. 1B). Shoots often emerged from the florets. The mode of regeneration appeared to be multiple shoot formation from multiple meristems followed by microtillering.

The shoots turned green in two days after transfer to fresh shoot regeneration (DM-8) medium under light. Shoot differentiation progressed simultaneously with proliferation of green, regenerated callus. (FIG. 1C). The subcultured callus showed various degrees of conversion to shoots.

Interestingly, the shoot regeneration medium could be used for shoot multiplication and production of complete plants as well. Small clusters of shoots, upon transfer to fresh medium, produced more shoots, and within one month further divisional was necessary. Many of the shoots elongated beyond 2 cm in length and developed roots (See Table 1), yielding 70-90 shoots per plate. The rate of shoot proliferation remained the same after subsequent cycles of subculture.

Shoot elongation varied, and it was necessary to separate shoots that elongated so much that it was impractical to subculture them in Petri dishes. The regenerating tissue was separated into two fractions: elongating shoots and initiating shoots.

TABLE 1
Effect of media on shoot regeneration and rooting of shoots (means ± SE
(n = 5).

| | NO. OF ELONGATED | |
|---|---|---|
| | SHOOTS PER GRAM | PERCENT SHOOTS |
| MEDIUM | TISSUE | WITH ROOTS |
| DM-8 | 19.6 ± 2.2 | 53 ± 2.4 |
| DM-5 | 21.8 ± 2.3 | 71 ± 5.7 |
| DM-3 light | 8.4 ± 1.2 | 56 ± 3.5 |
| DM-3 dark | 2.5 ± 0.3 | 55 ± 2.9 |

The elongating shoot clusters were transferred onto DM-5 medium in Magenta boxes with a low level of cytokinin (thidiazuron) where shoot proliferation continued. The DM-5 shoot proliferation medium could have also been used for shoot multiplication and production of complete plants. As on DM-8, small clusters of shoots produced more shoots upon transfer to fresh medium. Most of the shoots elongated beyond 5 cm in length and developed roots (See Table 1) yielding 80 to 100 shoots per Magenta box. The rate of shoot proliferation remained the same after subsequent cycles of subculture. Complete plantlets or shoot clusters separated from the regenerating callus developed a healthy root system and the leaves elongated on hormone-free medium in Magenta boxes.

The fraction of tissue with initiating shoots from DM-8 medium was cultured on lowered cytokinin-level DM-3 medium either in the dark or under light. Callus on DM-3 medium is green under light and is completely covered with short shoots, and retained its original regeneration capacity for at least 18 months. The majority of the shoots did not elongate but kept multiplying (See Table 1). The same medium can be used for regenerating callus maintenance in the dark. Callus is pale yellow in the dark (FIG. 1D), and shoot multiplication is dominant over elongation (Table 1).

DM-3 medium in the dark thus makes it possible to have a long-term regenerating callus culture and to avoid loosing the regenerating callus via complete conversion to shoots. Shoot regeneration can be easily effected by transferring portions of callus onto DM-8, DM-5, or hormone-free medium. Over 200 individual plants were established and grown under growth chamber conditions without difficulty.

Somatic embryos were not detected in the present A donax cultures under the conditions used. Without being bound by this or any other theory, it has been suggested that organization of single pole shoot meristems result from precocious germination of somatic embryos before complete development in graminoids which are characterized by regeneration occurring exclusively by somatic embryogenesis. See, e.g., Ozias-Akins, P. et al., Protoplasma, 110:417-420 (1982). However, the examples suggest that multiple shoot cultures can produce clones in high yield.

### EXAMPLE 2

This example illustrates the preparation of complete plantlets from excised A. donex cell tissue.

Shoot tips bulging with developing inflorescences were collected from a large, naturalized patch of giant reed. All but one leaf sheath were stripped carefully so as not to expose the inflorescence. Shoot tips were disinfected by shaking in a solution of 5 times diluted commercial bleach solution containing 10% (v/v) ethanol and 0.1% Tween 80 surfactant (w/v) for 15 min. Shoot tips were rinsed three times with sterile water.

The immature inflorescences were excised, chopped, and placed on the primary solid culture medium containing (in mg l⁻¹, unless indicated otherwise) MS (Murashige and Skoog, 1975) basal salts (Sigma Fine Chemicals) 4,300 (which are nutrient salts); Miller's salt solution (6% [w/v] KH₂PO₄), 3 ml; myo-inositol, 100; Vitamix (Marton and Browse, 1991), 2 ml; sucrose, 30,000, supplemented with the plant growth regulators adenine, 80; 2,4-dichlorophenoxyacetic acid, 0.2; and thidiazuron, 0.1, and solidified with Gellan gum (Phytagel brand from Sigma Fine Chemicals), 2000. The primary explants were incubated in the dark at 25° C. for four weeks.

In three to four weeks, callus formed at the tips of pedicels and inflorescence stem segments and from flower parts. The callus was white and more or less translucent, initially without shoots, but soon displaying signs of differentiation and pale yellowish color. This regenerating tissue culture could be maintained for at least 3 years by subculturing every four weeks on the primary culture medium in the dark.

The shoots turned green in two days after transfer to secondary culture medium for shoot regeneration and multiplication under light. Continuous illumination was used (30-50 μmol m⁻²s⁻¹; mixture of incandescent and cool white fluorescent tubes: Sylvania and Power-Twist Vita-Lite 40 W) at 25° C. Shoot differentiation progressed simultaneously with proliferation of green, regenerating callus. The subcultured callus showed various degrees of conversion to shoots.

The secondary medium contained (in mg l⁻¹; unless indicated otherwise) MS (Murashige and Skoog, 1975) basal salts (Sigma Fine Chemicals) 4,300; Miller's salt solution (6% [w/v] KH₂PO₄; sucrose, 30,000; supplemented with the plant hormone thidiazuron, 0.02, and solidified with Phytagel (Sigma Fine Chemicals), 2 g l⁻¹.

Small plantlets and clumps of multishoots were transferred to the tertiary culture medium, which differed from the secondary medium only in that it contained no hormones.

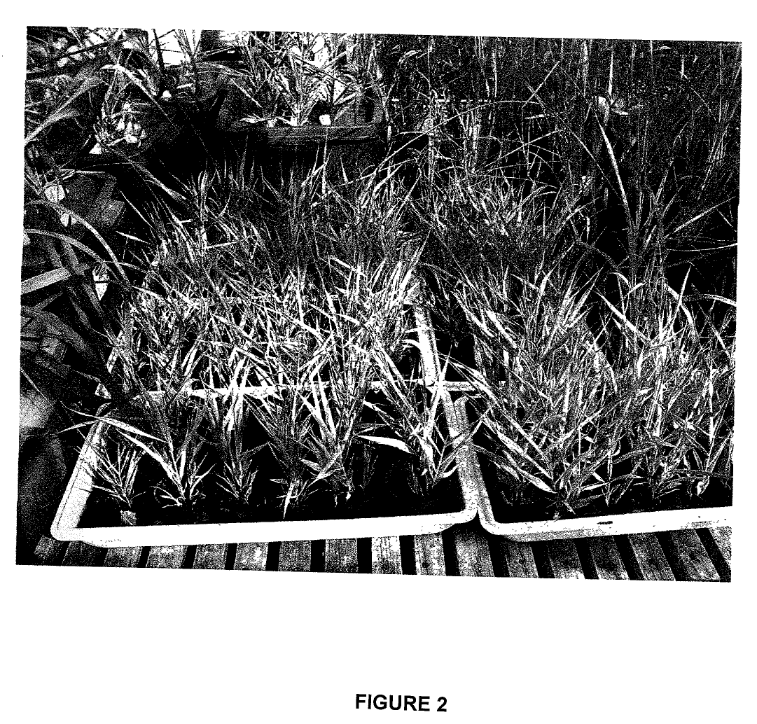

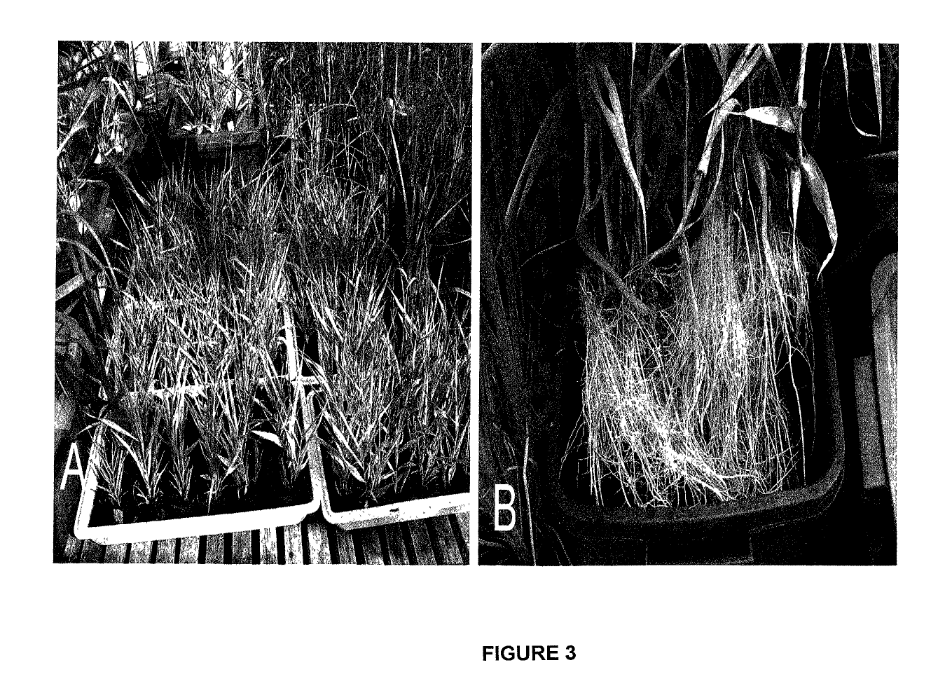

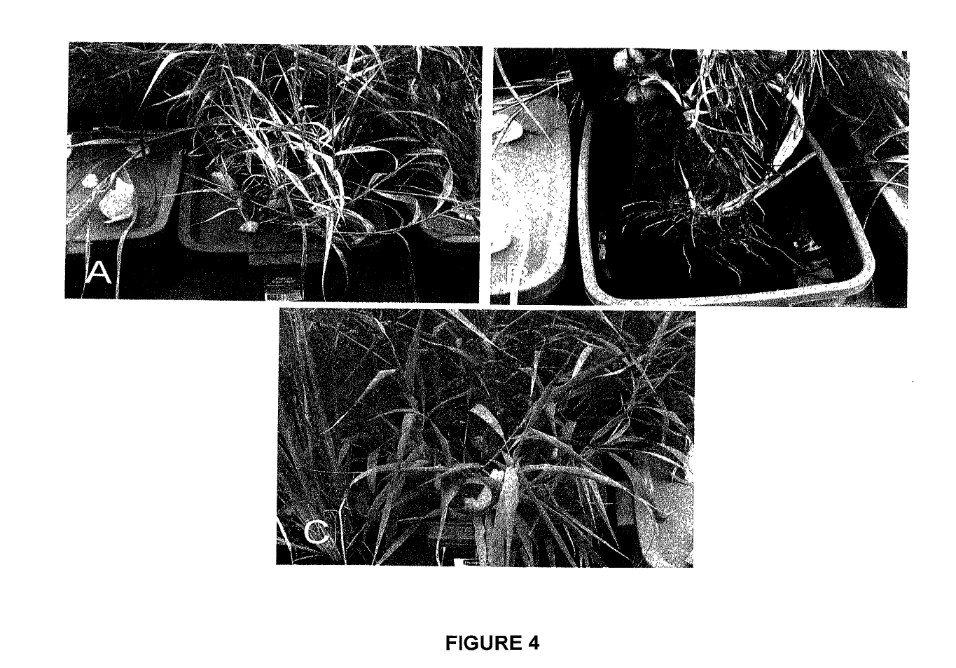

Elongated and rooted plants were separated, then potted in the greenhouse, and initially kept under plastic wrap cover for 5 days to help acclimatization. FIG. 2 shows a photograph of plants six weeks after they were transferred to potting soil. The plants shown are clones of A. donax that were grown by the present method from totipotent tissue culture tissue. Also shown in FIG. 2 is the extensive root system of A. donax plants grown in a standard liquid hydroponic medium.

The results of the observation for shoot formation after 4 weeks are shown in Table 2.

TABLE 2
Effect of media on shoot regeneration and rooting of shoots (means ± SE
(n = 5)).

| | No. of elongated | Percent shoots with |
|---|---|---|
| Medium | shoots per g tissue | roots |
| Primary | 8.4 ± 1.2 | 56 ± 3.5 |
| Secondary | 21.8 ± 2.3 | 71 ± 5.7 |

As evident from the table, both the number of the shoots formed from the tissue culture and the number shoots that developed roots increased upon transfer from the primary to the secondary medium.

### EXAMPLE 3

This example illustrates the transfer and expression of a heterologous gene into A. donax tissue by the present method.

Cross-sectional segment of immature A. donax inflorescence were prepared and cultivated as described in Example 2. The totipotent tissue was cocultivated with Agrobacterium tumefaciens carrying plasmid pMSF3022, which carried the bar gene for positive selection in plant cells. The gene confers resistance to the antibiotic/herbicide phosphinothricin.

Cocultivation was carried out in 6 ml. of liquid primary culture medium for four days in the dark at room temperature. Explants were then rinsed with liquid medium and placed on solid selective and non-selective control medium containing the antibiotic/herbicide phosphinothricine at 10 mg/l. All medium contained tidarcillin at 400 mg/l to eliminate residual A. tumefaciens. Controls include explants incubated without A. tumefaciens. The efficacy of the gene transfer (and proof of expression) can be seen in FIG. 3, which shows the development of herbicide resistant embryogenic tissue on explants cocultivated with Agrobacterium tumefaciens (FIG. 3C). This can be contrasted with control explants (which were not contacted with A. tumefaciens) that were killed by 10 mg/l of phosphinothricin (FIG. 3A). Also shown are control explants that have developed callus in the absence of phosphinothricin (FIG. 3B), and cocultivated explants that have developed callus in the absence of phosphinothricin (FIG. 3D).

It was concluded, therefore, that the transfer of a heterologous gene into totipotent tissue occurred, and also that the gene was expressed in the cloned plants that were products of the titopotent tissue.

### EXAMPLE 4

This example illustrates the operation of cloned plants of A. donax L. in a phytoreactor to cleanse organic waste materials from water.

Sustained titopotent cultures of A donax were grown as described in Example 2, and cloned plants derived from the titopotent tissues were established in a standard liquid hydroponic solution in plastic tubs (Shown in FIG. 4A). In one phytoreactor tub, trichloroethene (trichloroethylene) was added to a concentration of 0.25 mM. This was believed to be a high concentration of the organic material, because the allowable EPA concentration is 0.005 mg/l.

FIG. 4 illustrates the application of the cloned A. donax plants in the phytoreactor system, where (A) shows the upper part of plants in a phytoreactor container suspended in a standard hydroponic medium, (B) shows the roots of A. donax plants after challenge with 0.25 mM trichloroethene solution, and (C) shows the roots of control plants. After a recovery period of 3 to 4 weeks, the roots of the challenged plants fully recovered and appeared to be the same as the control plants as shown in (C).

This shows the ability of the cloned A. donax plants to serve in phytoreactors for the remediation of wastewaters.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained,

As various changes could be made in the above methods and compositions without departing from the scope of the invention, it is intended that all matter contained in the above description and shown in the accompanying drawing shall be interpreted as illustrative and not in a limiting sense.

What is claimed is:

1. A method for the production of totipotent tissue culture of giant reed (Arundo donax L.), the method comprising:
selecting an explant of living tissue from Arundo donax L.; and
cultivating the A. donax tissue on a primary medium to produce totipotent A. donax tissue.

2. The method according to claim 1, comprising, in addition:
cultivating the totipotent A. donax tissue on a secondary medium to produce complete plantlets having roots and shoots.

3. The method according to claim 2, wherein the explant of living tissue from Arundo donax L. is cut into cross-sectional segments before cultivation on a primary medium.

4. The method according to claim 3, wherein the primary medium comprises a plant hormone and is capable of supporting the multiplication of the totipotent tissue.

5. The method according to claim 4, wherein the secondary medium comprises a plant hormone and is capable of supporting the multiplication of the growing tissue by microtillering.

6. The method according to claim 5, comprising, in addition:
cultivation on a tertiary medium which is free of added plant hormones and which supports shoot elongation.

7. The method according to claim 3, wherein the explant comprises an inflorescence.

8. The method according to claim 7, wherein the inflorescence is from a pre-flowering shoot with leaf sheaths completely enclosing the developing but still unemerged immature inflorescence.

9. The method according to claim 2, wherein the primary medium and the secondary medium are solidified by the addition of a gelling agent that is selected from the group consisting of agar, agarose, Gellan gum, gelcarin, and mixtures thereof.

10. The method according to claim 1, wherein the primary culture medium is a mineral nutrient medium supplemented with plant hormones, vitamins and a carbohydrate or a mixture of carbohydrates

11. The method according to claim 10, wherein sucrose is present in a concentration of about 30 g/l.

12. The method according to claim 6, wherein the secondary medium and the tertiary medium are supplemented with sucrose.

13. The method according to claim 12, wherein the sucrose is present in a concentration of about 30 g/l.

14. The method according to claim 10, wherein the plant hormone of the primary medium comprises an auxin and a cytokinin.

15. The method according to claim 14, wherein the auxin comprises 2,4-dichlorophenoxyacetic acid and the cytokinin comprises thidiazuron.

16. The method according to claim 2, wherein the plant hormone of the secondary medium comprises a cytokinin.

17. The method according to claim 16, wherein the cytokinin comprises thidiazuron.

18. The method according to claim 6, comprising the introduction of a heterologous gene into the Arundo donax L. tissue.

19. The method according to claim 18, wherein the introduction of a heterologous gene is effected by cocultivation with Agrobacterium tumefaciens that results in the transfer of one or more genes from A. tumefaciens to the A. donax tissue.

20. The method according to claim 18, wherein the introduction of a heterologous gene is effected by DNA transfer.

21. A method for the micropropagation of giant reed (Arundo donax L.), the method comprising:
selecting an explant of living tissue from Arundo donax L.;
cultivating the A. donax tissue on a primary medium to produce a totipotent tissue culture;
cultivating the totipotent A. donax totipotent tissue on a secondary medium to produce complete plantlets having roots and shoots; and
acclimating the plantlets in soil.

22. The method according to claim 21, comprising the introduction of a heterologous gene into the Arundo donax L. totipotent tissue.

23. The method according to claim 22, wherein the plantlets are transgenic plantlets and the plantlets are used for phytoremediation or in phytoreactors.

24. Totipotent Arundo donax tissue that is produced by the method of claim 1.

25. Transgenic totipotent Arundo donax tissue that is produced by the method of claim 18.

26. A plant of Arundo donax L. that is produced by the method of claim 21.

27. A transgenic plant of Arundo donax L. that is produced by the method of claim 22.

28. A method for removal of an environmental pollutant from wastewater, the method comprising:
providing at least 10 A. donax plants that possess the same genetic characteristics;
establishing the plants in a liquid medium; and
contacting the roots of the plants in the liquid medium with an environmental pollutant,
thereby causing the environmental pollutant to be removed from the liquid medium.

29. The method according to claim 28, wherein at least 1000 A. donax plants that possess the same genetic characteristics are provided.

30. A method for bioremediation of an environmental pollutant from a land area, the method comprising:
providing at least 10 A. donax plants that possess the same genetic characteristics;
establishing the plants in soil;
and contacting the roots of the plants with the environmental pollutant in the land area,
thereby causing the environmental pollutant to be removed from the land area.

31. The method according to claim 30, wherein at least 1000 A. donax plants that possess the same genetic characteristics are provided.
